(19) United States
(12) Patent Application Publication
(10) Pub. No.: US 2025/0144317 A1
(43) Pub. Date: May 8, 2025

(54) NEEDLE COVER MANAGEMENT AND NEEDLE OBSCURING DEVICES FOR INJECTION SYSTEMS AND METHODS

(71) Applicant: CREDENCE MEDSYSTEMS, INC., Menlo Park, CA (US)

(72) Inventor: Stephen H. Diaz, Palo Alto, CA (US)

(73) Assignee: CREDENCE MEDSYSTEMS, INC., Menlo Park, CA (US)

(21) Appl. No.: 18/939,157

(22) Filed: Nov. 6, 2024

Related U.S. Application Data
(60) Provisional application No. 63/596,404, filed on Nov. 6, 2023.

(51) Int. Cl. A61M 5/32

(52) U.S. Cl. CPC A61M 5/3232; A61M 5/3293

(57) ABSTRACT

A system for injection includes a syringe body having a distal end and a syringe interior. The system also includes a stopper member disposed in the syringe interior, and a plunger member coupled to the stopper member and configured to be manipulated to insert the stopper member distally in the syringe interior relative to the syringe body. The system further includes a needle hub assembly coupled to the syringe body at the distal end thereof and including a needle hub, and a needle coupled to the needle hub. Moreover, the system includes a needle obscuring assembly removably coupled to the needle hub over the needle, the needle obscuring member configured to be disposed at least partially over the needle. The system also includes a safe retraction system configured to retract such that a distal tip of the needle is disposed within the needle hub or the syringe body.

The present application claims priority to U.S. Provisional Patent Application Ser. No. 63/596,404, filed on Nov. 6, 2023 under attorney docket number CM.30043.00 and entitled “NEEDLE COVER MANAGEMENT AND NEEDLE OBSCURING DEVICES FOR INJECTION SYSTEMS AND METHODS.” The present application includes subject matter similar to the subject matter described in the following co-owned U.S. patent applications: (1) U.S. patent application Ser. No. 14/321,706, filed Jul. 1, 2014 and issued as U.S. U.S. Pat. No. 9,814,842 on Nov. 14, 2017 under attorney docket number CM.20001.00 and entitled “SAFETY SYRINGE”; (2) U.S. patent application Ser. No. 14/543,787, filed Nov. 17, 2014 and issued as U.S. Pat. No. 10,300,217 on May 28, 2019 under attorney docket number CM.20002.00 and entitled “SYSTEM AND METHOD FOR DRUG DELIVERY WITH A SAFETY SYRINGE”; (3) U.S. patent application Ser. No. 14/696,342, filed Apr. 24, 2015, and issued as U.S. Pat. No. 10,010,677 on Jul. 7, 2018 under attorney docket number CM.20003.00 and entitled “SYSTEM AND METHOD FOR SAFETY SYRINGE”; (4) U.S. patent application Ser. No. 15/801,239, filed on Nov. 1, 2017 and issued as U.S. Pat. No. 10,926,038 on Feb. 23, 2021 under attorney docket number CM.20011.00 and entitled “SYSTEM AND METHOD FOR SAFETY SYRINGE”; (5) U.S. patent application Ser. No. 15/801,259, filed on Nov. 1, 2017, and issued as U.S. Pat. No. 10,864,330 on Dec. 15, 2020 under attorney docket number CM.20012.00 and entitled “SYSTEM AND METHOD FOR SAFETY SYRINGE”; (6) U.S. patent application Ser. No. 15/801,281 filed on Nov. 1, 2017 and issued as U.S. Pat. No. 10,912,894 on Feb. 9, 2021 under attorney docket number CM.20013.00 and entitled “CARTRIDGE SAFETY INJECTION SYSTEM AND METHODS”; (7) U.S. patent application Ser. No. 15/801,304 filed on Nov. 1, 2017 and issued as U.S. Pat. No. 10,960,144 on Mar. 30, 2021 under attorney docket number CM.20015.00 and entitled “SYSTEM AND METHOD FOR SAFETY SYRINGE”; (8) U.S. patent application Ser. No. 16/435,429 filed on Jun. 7, 2019 under attorney docket number CM.20019.00 and entitled “SYSTEM AND METHOD FOR SAFETY SYRINGE”; (9) U.S. patent application Ser. No. 16/798,188, filed on Feb. 21, 2020 under attorney docket number CM.20023.00 and entitled “SYSTEM AND METHOD FOR SAFETY SYRINGE”; (10) U.S. patent application Ser. No. 16/837,835, filed Apr. 1, 2020 under attorney docket number CM.20025.00 and entitled “POLYMERIC INJECTION SYSTEMS”; (11) U.S. patent application Ser. No. 16/908,531 filed on Jun. 22, 2020 under attorney docket number CM.20026.00 and entitled “INJECTION SYSTEM AND METHOD”; (12) U.S. patent application Ser. No. 17/031,108 filed on Sep. 24, 2020 under attorney docket number CM.20027.00 and entitled “SYSTEM AND METHOD FOR SAFETY SYRINGE”; (13) U.S. patent application Ser. No. 17/364,546 filed on Jun. 30, 2021 under attorney docket number CM.20028.00 and entitled “SYSTEM AND METHOD FOR SAFETY SYRINGE”; (14) U.S. Provisional Patent Application Ser. No. 63/094,313 filed on Oct. 20, 2020 under attorney docket number CM.30030.00 and entitled “RETRACTION MECHANISM FOR SAFE INJECTION SYSTEM”; (15) U.S. Provisional Patent Application Ser. No. 63/300,394, filed on Jan. 18, 2022 under attorney docket number CM.30034.00 and entitled “INJECTION SYSTEM AND METHOD”; (16) U.S. patent application Ser. No. 18/300,125, filed on Apr. 13, 2023 under attorney docket number CM.20035.00 and entitled “NEEDLE COVER MANAGEMENT AND NEEDLE HIDING DEVICES FOR INJECTION SYSTEMS AND METHODS”. The contents of the applications and patents identified herein are fully incorporated herein by reference as though set forth in full.

### FIELD OF THE DISCLOSURE

The present disclosure relates generally to injection systems, devices, and processes for facilitating various levels of control over injection processes, and more particularly to devices and methods related to obscuring, concealing, or hiding a needle in injection systems, with or without safety features distinct from the needle cover handling devices, in healthcare environments.

### BACKGROUND

Millions of syringes, such as that depicted in FIG. 1A (2), are consumed in healthcare environments every day. A typical syringe (2) comprises a tubular body (4), a plunger (6), and an injection needle (8). As shown in FIG. 1B, such a syringe (2) may be utilized not only to inject fluid into a patient, but also to withdraw or expel fluid out of or into a container such as a medicine bottle, vial, bag, or other drug containment system (10). Indeed, due to regulatory constraints in some countries such as the United States as well as sterility maintenance concerns, upon use of a medicine bottle (10) with a syringe (2) as shown in a particular patient's environment, such medicine bottle may only be utilized with a single patient and then must be disposed of—causing significant medical waste from bottle and remaining medicine disposal, and even contributing to periodic shortages of certain critical drugs. Referring to FIG. 2A, three Luer-type syringes (12) are depicted, each having a Luer fitting geometry (14) disposed distally, so that they may be coupled with other devices having similar mating geometry, such as the Luer manifold assembly (16) depicted in FIG. 2B. The Luer manifold assembly of FIG. 2B may be used to administer liquid drugs to the patient intravenously with or without the use of an intravenous infusion bag. The Luer fittings (14) of the syringes of FIG. 2A may be termed the “male” Luer fittings, while those of FIG. 2B (18) may be termed the “female” Luer fittings; one of the Luer interfaces may be threaded (in which case the configuration may be referred to as a “Luer lock” configuration) so that the two sides may be coupled by relative rotation, which may be combined with compressive loading. In other words, in one Luer lock embodiment, rotation, possibly along with compression, may be utilized to engage threads within the male fitting (14) which are configured to engage a flange on the female fitting (18) and bring the devices together into a fluid-sealed coupling. In another embodiment, tapered interfacing geometries may be utilized to provide for a Luer engagement using compression without threads or rotation (such a configuration may be referred to as a “slip-on” or “conical” Luer configuration). While such Luer couplings are perceived to be relatively safe for operators, there is risk of medicine spilling/leaking and parts breakage during assembly of a Luer coupling. The use of needle injection configurations, on the other hand, carries with it the risk of a sharp needle contacting or stabbing a person or structure that is not desired. For this reason, so called “safety syringes” have been developed.

One embodiment of a safety syringe (20) is shown in FIG. 3, wherein a tubular shield member (22) is spring biased to cover the needle (8) when released from a proximal/retracted position relative to the syringe body (4). The tubular needle shield (22) is “locked” in the distal/extended configuration, such that the needle shield (22) can no longer be returned to the proximal/retracted position, to prevent accidental needle sticks after injection.

Another embodiment of a safety syringe (24) is shown in FIGS. 4A-4B. With such a configuration, after full insertion of the plunger (6) relative to the syringe body (4), the retractable needle (26) is configured to retract (28, 26) back to a safe position within the tubular body (4), as shown in FIG. 4B. Such a configuration which is configured to collapse upon itself may be associated with blood spatter/aerosolization problems, the safe storage of pre-loaded energy which may possibly malfunction and activate before desirable, loss of accuracy in giving full-dose injections due to residual dead space within the spring compression volume, and/or loss of retraction velocity control which may be associated with pain and patient anxiety.

Further complicating the syringe marketplace is an increasing demand for prefilled syringe assemblies such as those depicted in FIGS. 5A and 5B, which generally comprise a syringe body, or “drug enclosure containment delivery system”, (34), a plunger tip, plug, or stopper (36), and a distal seal or cap (35) which may be fitted over a Luer type interface (FIG. 5A shows the cap 35 in place; FIG. 5B has the cap removed to illustrate the Luer interface 14). Liquid medicine may reside in the volume, or medicine reservoir, (40) between the distal seal and the distal end (37) of the plunger tip (36). The plunger tip (36) may comprise a standard butyl rubber material and may be coated, such as with a biocompatible lubricious coating (e.g., polytetrafluoroethylene (“PTFE”)), to facilitate preferred sealing and relative motion characteristics against the associated syringe body structure and material. The proximal end of the syringe body (34) in FIG. 5B comprises a conventional integral syringe flange (38), which is formed integral to the material of the syringe body (34). The flange (38) is configured to extend radially from the syringe body (34) and may be configured to be a full circumference, or a partial circumference around the syringe body (34). A partial flange is known as a “clipped flange” while the other is known as a “full flange.” The flange is used to grasp the syringe with the fingers to provide support for pushing on the plunger to give the injection. The syringe body (34) preferably comprises a translucent material such as a glass or polymer. To form a contained volume within the chamber or reservoir (40), and to assist with expulsion of the associated fluid through the needle, a plunger tip (36) may be positioned within the syringe body (34). The syringe body (34) may define a substantially cylindrical shape (i.e., so that a plunger tip 36 having a circular cross-sectional shape may establish a seal against the syringe body (34)), or be configured to have other cross-sectional shapes, such as an ellipse.

Such assemblies are desirable because they may be standardized and produced with precision in volume by the few manufacturers in the world who can afford to meet all of the continually changing regulations of the world for filling, packaging, and medicine/drug interfacing materials selection and component use. Such simple configurations, however, generally will not meet the new world standards for single-use, safety, auto-disabling, and visually obscuring, concealing, or hiding the needle in injection systems. Thus, certain suppliers have moved to more “vertical” solutions, such as that (41) featured in FIG. 5C, which attempts to meet all of the standards, or at least a portion thereof, with one solution; as a result of trying to meet these standards for many different scenarios, such products may have significant limitations (including some of those described above in reference to FIGS. 3-4B) and relatively high inventory and utilization expenses.

Most injection systems include a needle cover (e.g., rigid needle shield, needle shield, etc.) removably coupled to a needle hub and configured to cover the sharp distal end of the needle to minimize accidental needle sticks, to protect the needle from mechanical damage, and to seal the injection system before use. Existing injection systems are configured such that needle covers are removed manually by a user's hands. Needle covers are secured to needle hubs. Significant force must be exerted by the user's hands to remove needle covers from needle hubs. The user's hands are proximate the sharp distal end of the needle when removing the needle cover. As a result of this proximity, the removal of the needle cover can result in accidental needle sticks (e.g., with a bounce back of the hand grasping the needle cover or movement of the hand holding the injection system). When a user applies force to the proximal end of the needle cover to avoid needle sticks, the needle cover may be launched from the needle hub and end up as unwanted trash on the floor of the medical facility. Accordingly, there exists a need for needle cover handling devices for a more controlled and safer removal of needle covers from injection systems.

Further, for multiple chamber injection systems such as those described in U.S. patent application Ser. No. 15/801,259, which was previously incorporated by reference herein, pressure may build up in a distal chamber when mixing drug components therein prior to injection with the distal end of the needle sealed by a needle cover. Additionally, the needle may be fixedly or removably mounted to a flange on a cartridge body instead of a syringe. Such cartridge injection systems are disclosed in U.S. patent application Ser. No. 15/801,281, which was previously incorporated by reference herein.

Accordingly, there exists a need for needle handling devices to removably hold a needle cover apart from but the injection system but still visually obscuring, concealing, or hiding the needle. In particular, there is a need for needle cover handling devices, which may utilize the existing and relatively well-controlled supply chain of conventionally delivered syringe assemblies.

### SUMMARY

Embodiments are directed to injection systems. In particular, the embodiments are directed to needle cover handling devices and needle obscuring assemblies for injection systems.

In one embodiment, a system for injection includes a syringe body having proximal and distal ends and defining a syringe interior. The system also includes a stopper member disposed in the syringe interior. The system further includes a plunger member coupled to the stopper member and configured to be manipulated to insert the stopper member distally in the syringe interior relative to the syringe body. Moreover, the system includes a needle hub assembly coupled to the syringe body at the distal end thereof and including a needle hub, and a needle coupled to the needle hub. The system also includes a needle obscuring assembly removably coupled to the needle hub over the needle, the needle obscuring assembly including a liner coupled to the needle hub, and a needle obscuring member slidably disposed on and movable relative to the liner, the needle obscuring member configured to be disposed at least partially over the needle. The system further includes a safe retraction system configured to retract the needle at least partially within the syringe body after an injection with the system is completed such that a distal tip of the needle is disposed within the needle hub or the syringe body.

In one or more embodiments, the needle hub includes a compression ring, the compression ring defines an annular surface. In addition, the liner includes a flexible latch configured to engage the annular surface of the compression ring and resist removal of the liner from the needle hub. Additionally, the latch of the liner is further configured to secure the needle obscuring assembly to the needle hub.

In one or more embodiments, the needle obscuring assembly includes an extended configuration and a retracted configuration, where the needle obscuring member is moved distally relative to the liner, substantially obscuring the needle, when the needle obscuring assembly is in the extended configuration, and where the needle obscuring member is moved proximally relative to the liner at least partially exposing the needle, when the needle obscuring assembly is in the retracted configuration.

In one or more embodiments, the liner includes a pair of protrusions, where the needle obscuring member defines a pair of slots, and where each protrusion is partially disposed within a respective slot and configured to interfere with a proximally facing surface of the needle obscuring member defining the respective slot to limit proximal movement of the needle obscuring member relative to the liner when the needle obscuring assembly is moved into the retracted configuration.

In one or more embodiments, each protrusion of the liner is further configured to interfere with a distally facing surface of the needle obscuring member defining the respective slot to limit distal movement of the needle obscuring member relative to the liner when the needle obscuring assembly is moved into the extended configuration.

In one or more embodiments, the needle obscuring member includes a proximal end, a tubular body, a distal portion, where the needle obscuring member defines a lumen extending between the proximal end and the distal portion, and where the tubular body includes a plurality of inwardly extending movable members. In addition, the plurality of inwardly extending movable members includes a first pair of inwardly extending movable members and a second pair of inwardly extending movable members. Optionally, the first pair and second pair of inwardly extending movable members of the needle obscuring member are each movable radially inward and outward from the tubular body of the needle obscuring member.

In one or more embodiments, the system further includes a needle cover removably disposed within the lumen of the needle obscuring member and over the needle, where the needle cover includes a body surface and a proximally facing flange, and where the first pair of the inwardly extending movable members of the needle obscuring member is configured to contact the body surface of the needle cover and the second pair of the inwardly extending movable members of the needle obscuring member is configured to contact the proximally facing flange of the needle cover.

In one or more embodiments, the needle obscuring member is configured to move the needle cover distally when the needle obscuring assembly is moved distally into the extended configuration and exerts a distally directed force on the needle cover through the second pair of the inwardly extending movable members of the needle obscuring member to the proximally facing flange of the needle cover.

In one or more embodiments, the first pair of the inwardly extending movable members of the needle obscuring member is configured to exert a resistive force on the body surface of the needle cover to removably hold the position of the needle cover relative to the needle obscuring assembly, when the needle obscuring assembly is moved into the extended configuration. In addition, the resistive force exerted on the needle cover by the first pair of the inwardly extending movable members of the needle obscuring member is configured to be overcome by a user's fingers to remove the needle cover from the needle obscuring assembly. Additionally, moving the needle cover distally as the needle obscuring assembly is moved into the extended configuration reduces a force required to remove the needle cover from the needle hub.

In one or more embodiments, the first pair of inwardly extending movable members of the needle obscuring member is configured to move radially outward from the tubular body of the needle obscuring member when the needle obscuring assembly is in the retracted configuration to partially expose the needle.

In one or more embodiments, the second pair of inwardly extending movable members of the needle obscuring member is configured to move radially inward from the tubular body of the needle obscuring member when the needle obscuring assembly is in the retracted configuration to partially expose the needle.

In one or more embodiments, the each of the pair of slots of the needle obscuring member include a length configured to limit exposure of a predetermine length of the needle when the needle obscuring assembly is in the retracted configuration. In addition, the predetermined length of the needle is in a range between 25 to 6 millimeters. In some embodiments, the predetermined length of the needle is about 8 millimeters.

In one or more embodiments, the needle obscuring member defines an annular space, and the liner is at least partially disposed in the annular space.

In one or more embodiments, the needle obscuring member defines an outwardly sloping surface configured to facilitate application of a distally directed force to the needle obscuring member.

In one or more embodiments, the distal portion of the needle obscuring member prevents a distal tip of the needle from being seen from proximal of the needle obscuring member when the needle obscuring assembly is in the extended configuration.

In one or more embodiments, the distal portion of the needle obscuring member is configured to contact a patient's skin before a distal tip of the needle contacts the patient's skin during an injection using the system when needle obscuring assembly is in the extended configuration. In addition, the distal portion of the needle obscuring member defines a substantially flat distally facing surface. In some embodiments, the distal portion of the needle obscuring member includes a plurality of protrusions or cones on a distally facing surface thereof.

In one or more embodiments, the distal portion of the needle obscuring member includes a diameter larger than the diameter of the tubular body, and the distal portion further includes a distally facing surface. In addition, the distally facing surface includes a plurality of sensory masking elements or protrusions.

In one or more embodiments, the system further includes a safe retraction system configured to retract the needle at least partially within the syringe body after an injection with the system is completed such that the distal tip of the needle is disposed within the needle hub or the syringe body.

In one or more embodiments, the needle obscuring assembly includes an extended configuration and a retracted configuration. The liner and the needle obscuring member substantially overlap, and the liner is aligned with the proximal end of the needle obscuring member when the needle obscuring assembly is in the extended configuration. Moreover, the needle shield is moved distally relative to the liner and the liner is not aligned with the proximal end of the needle shield when the needle obscuring assembly is in the retracted configuration. In addition, the needle obscuring assembly is configured to move from a fully extended configuration to a fully retracted configuration. In some embodiments, the needle obscuring assembly is configured to move from a fully extended configuration to a partially retracted configuration. Optionally, the needle obscuring assembly is further configured to move from the fully retracted configuration into the fully extended configuration. Additionally, the needle obscuring assembly comprises no locking mechanism to lock the assembly in the extended configuration or in the retracted configuration.

The aforementioned and other embodiments of the invention are described in the Detailed Description which follows.

### BRIEF DESCRIPTION OF THE DRAWINGS

FIGS. 1A to 5C illustrate various aspects of conventional injection syringe configurations.

FIG. 6 is a perspective view of an injection system having a needle obscuring assembly and a needle cover described herein.

FIG. 7 is a perspective exploded view of the injection system of FIG. 6 illustrating various aspects of the needle obscuring assembly.

FIG. 8 is a perspective exploded view of a needle obscuring assembly for use with an injection system according to some embodiments.

FIG. 8B is a perspective view of a needle obscuring member disposed radially outside of a liner of the needle obscuring assembly of FIG. 8A.

FIG. 9A is a side view of the needle obscuring assembly of FIG. 8A.

FIG. 9B is a side and perspective the injection system needle obscuring assembly and the needle cover of FIG. 6.

FIGS. 10A-10B are perspective partially cross-sectional views of the needle obscuring assembly coupled to a needle hub, according to some embodiments.

FIGS. 10C-10E are perspective partially cross-sectional views of the needle obscuring assembly interface with a needle cover, according to some embodiments.

FIG. 10F is a perspective partially cross-sectional view of the needle obscuring assembly in an extended configuration without the needle cover, according to some embodiments.

FIGS. 10G-10H are perspective views of the needle obscuring assembly interface in a retracted configuration exposing a needle, according to some embodiments, and

FIG. 11 is a perspective view of an alternative needle obscuring assembly, according to some embodiments.

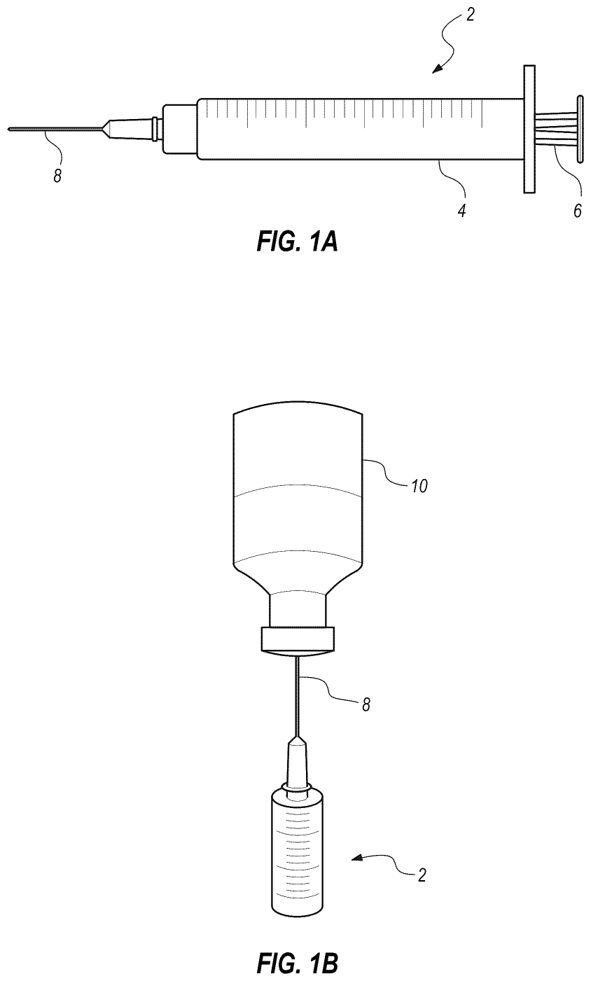

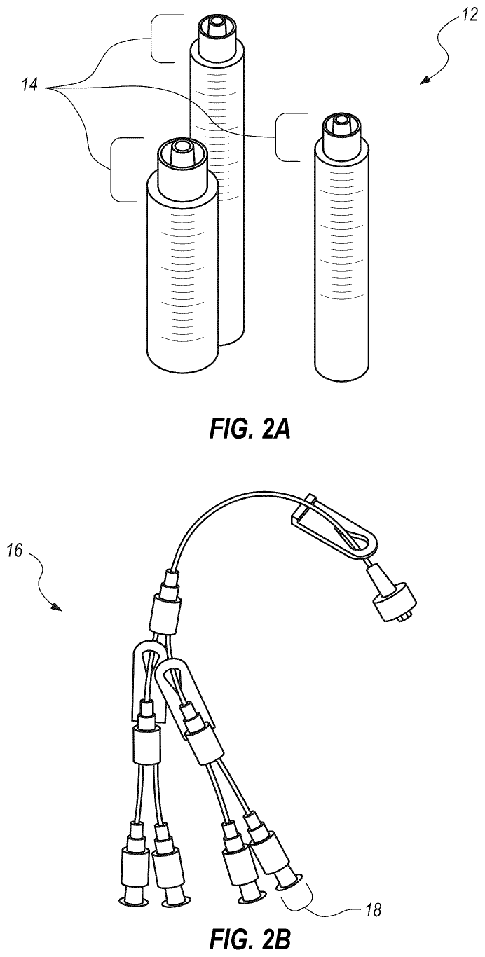

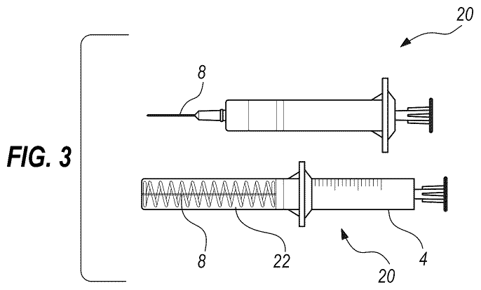

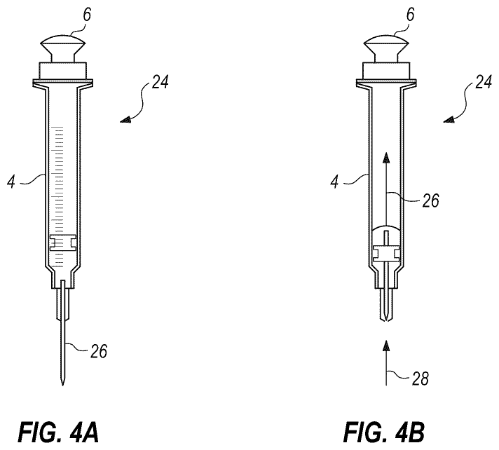

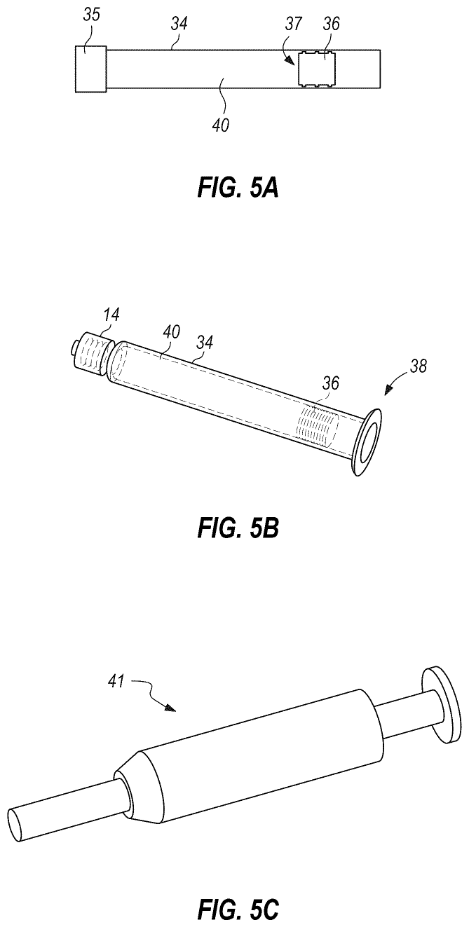

In order to better appreciate how to obtain the above-recited and other advantages and objects of various embodiments, a more detailed description of embodiments is provided with reference to the accompanying drawings. It should be noted that the drawings are not drawn to scale and that elements of similar structures or functions are represented by like reference numerals throughout. It will be understood that these drawings depict only certain illustrated embodiments and are not therefore to be considered limiting of scope of embodiments.

### DETAILED DESCRIPTION OF ILLUSTRATED EMBODIMENTS

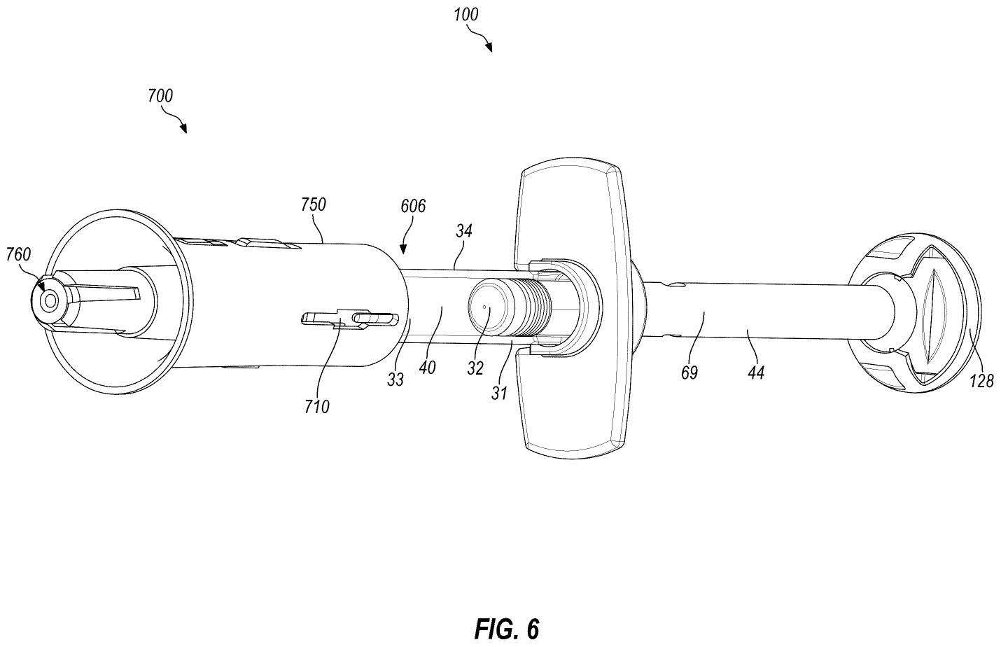

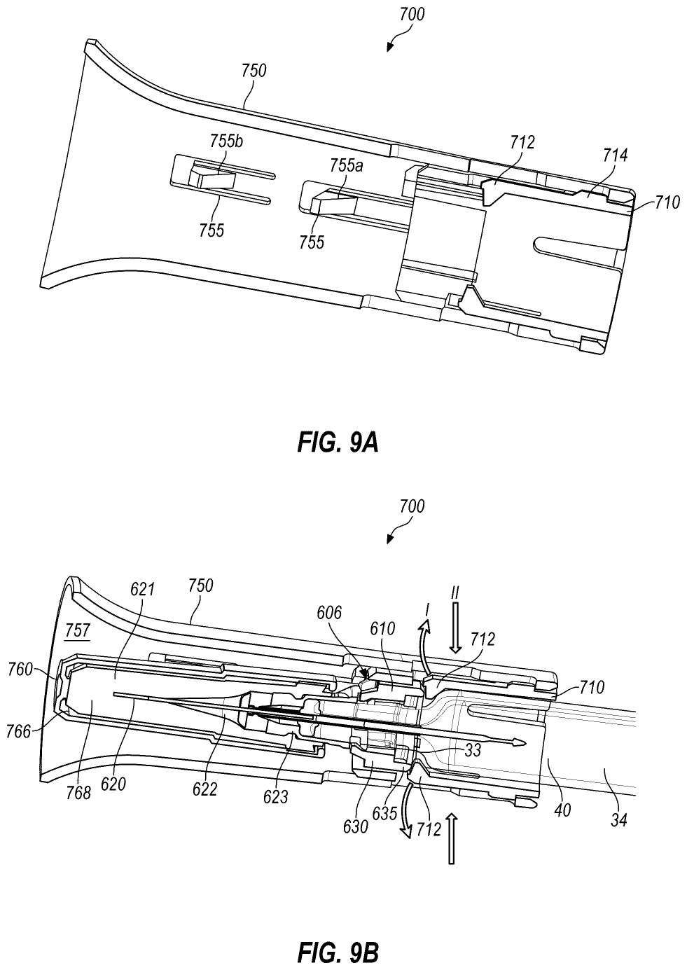

FIG. 6 depicts an injection system (100) having a needle obscuring assembly (700) and a needle cover (760), according to embodiments of the inventions. The injection system (100) includes a conventional off-the-shelf pre-filled syringe body (34) having a proximal end (31) and a distal end (33) defining a medicine reservoir or syringe interior (40). The medicine reservoir or syringe interior (40) is in fluid communication with a syringe needle (620), as shown in FIG. 9B. While the injection system (100) is a single chamber injection system, the needle obscuring assembly (700) can be utilized with multiple chamber injection systems, such as those described in U.S. patent application Ser. No. 18/300,125, which was previously incorporated by reference herein.

As shown in FIG. 9B, the injection system (100) includes a needle hub assembly (606) disposed at the distal end (33) of the syringe body (34). The needle hub assembly (606) includes a needle hub (610) and the needle (620), where the needle (620) is coupled to the needle hub (610). The needle hub (610) includes a compression ring (630), the compression ring (630) defines an annular surface (635). The compression ring (630) and/or the annular surface (635) have a larger diameter than the diameter of the syringe body (34), as shown in FIG. 9B. The needle (620) is covered by a needle cover (760) for storage and before use of the injection system (100) in a patient.

Referring back to FIG. 6, the injection system (100) further includes a plunger member (44) coupled to the proximal end (31) of the syringe body (34). The plunger member (44) includes the stopper member (32), a plunger housing member (69) and a plunger manipulation interface (128). The plunger member (44) is configured to be manipulated to insert the stopper member (32) distally in the syringe interior (40) relative to the syringe body (34) to deliver liquid medicine disposed within the syringe interior (40) into the patient via the needle (620). The plunger tip (32) may comprise a standard butyl rubber and may be coated, such as with a biocompatible lubricious coating (e.g., PTFE), to facilitate sealing and relative motion characteristics against the associated syringe body structure and material. Medicine delivery to the patient by the injection system (100) is controlled by distal insertion of the plunger member (44) relative to the syringe body (34) to various degrees by a user (e.g., sequential insertion), when the needle (620) is inserted into the patient.

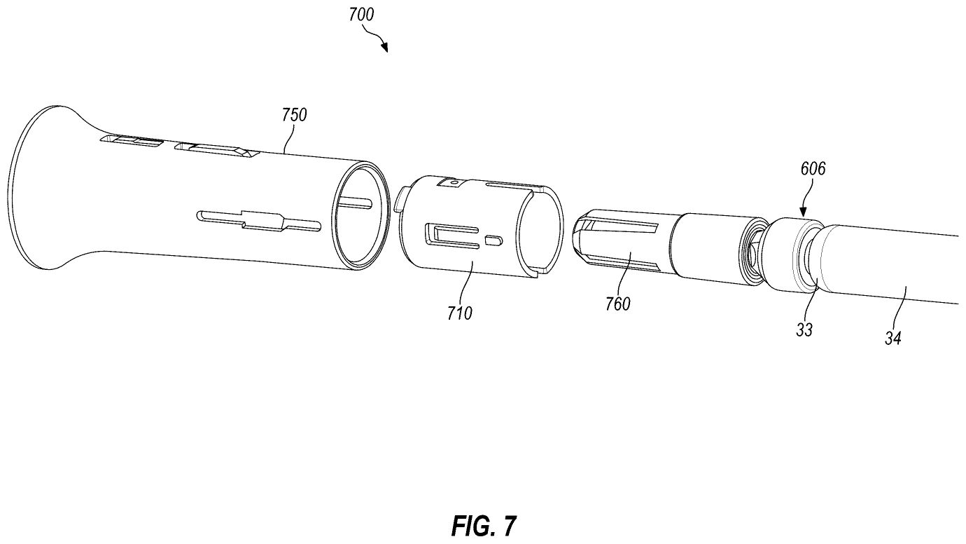

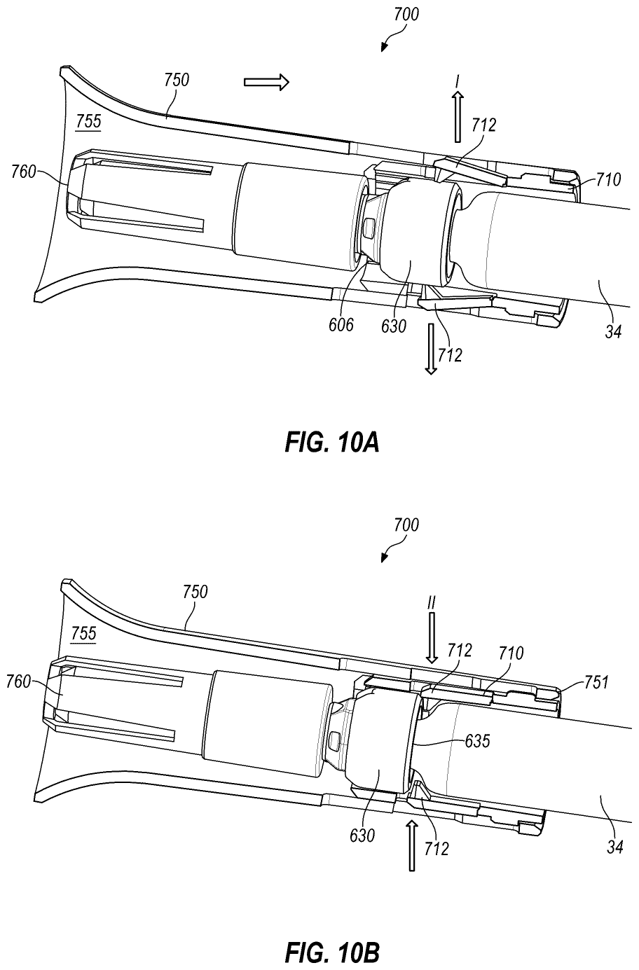

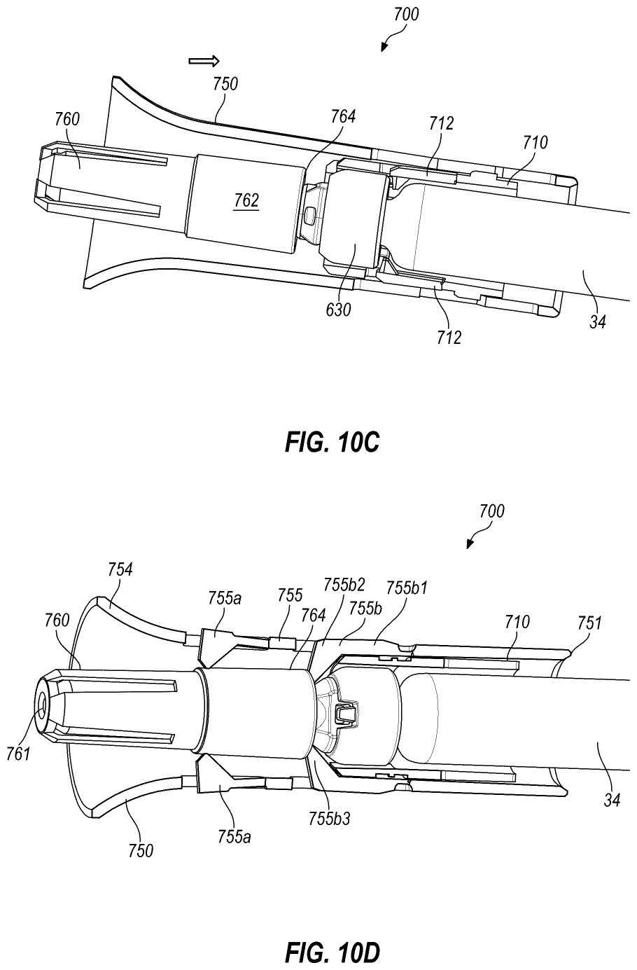

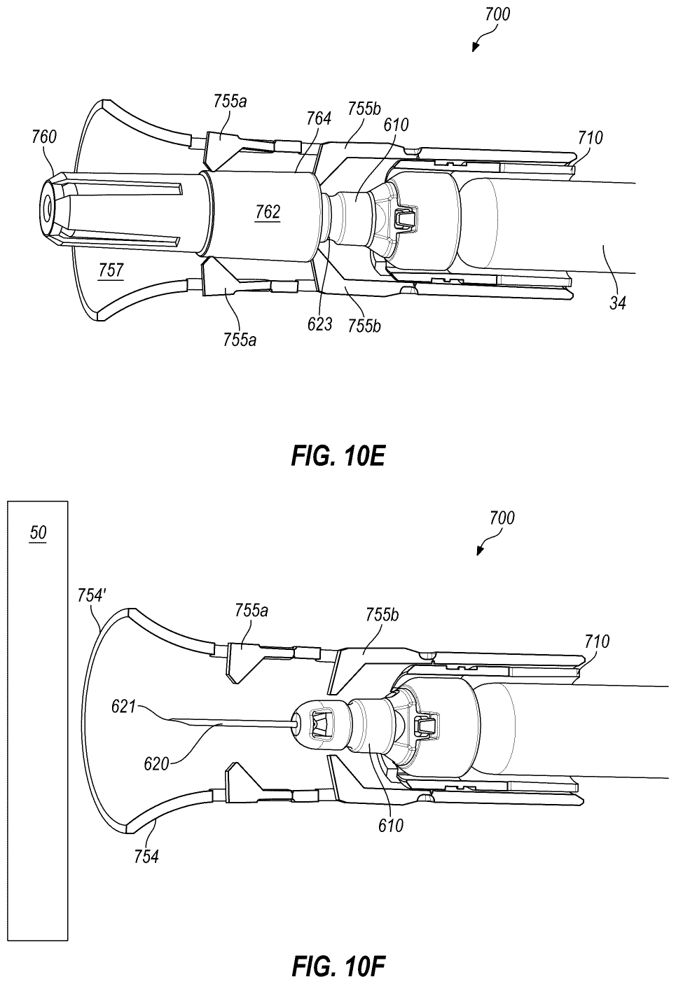

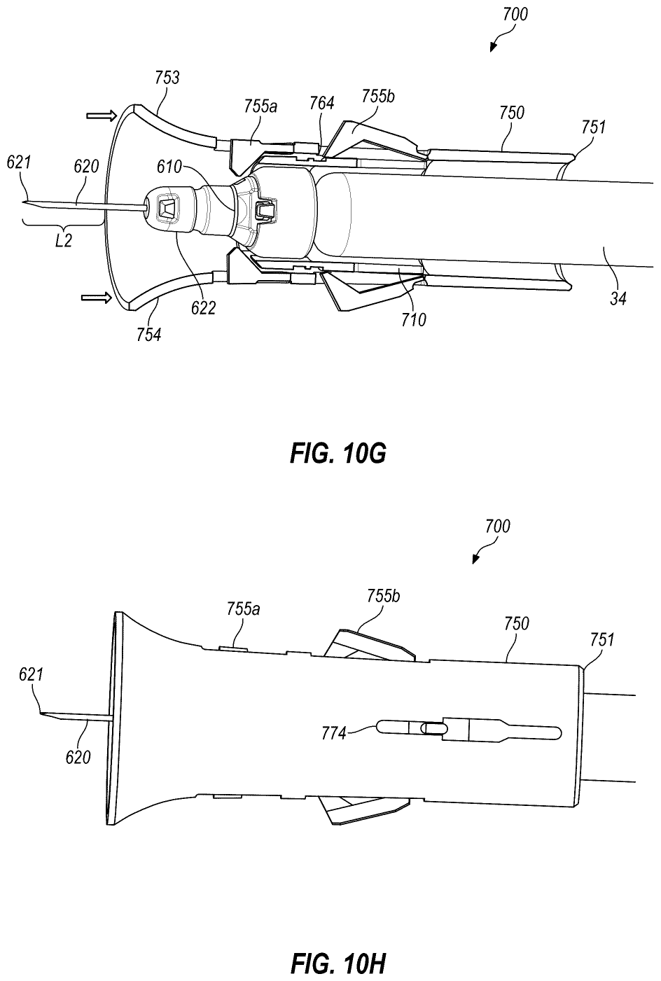

FIG. 7 illustrates an exploded view of the injection system (100) depicting the needle hub assembly (606) coupled to the distal end (33) of the syringe body (34), the needle cover (760), the liner (710) and the needle obscuring member (750). As shown in FIGS. 6-8B, the injection system (100) includes the needle obscuring assembly (700). The needle obscuring assembly (700) is removably coupled to the needle hub (610) over the needle (620). The needle obscuring assembly (700) includes a liner (710) coupled to the needle hub (610), and a needle obscuring member (750) slidably disposed on and movable relative to the liner (710). The needle obscuring member (750) is configured to translate and/or longitudinally moved (e.g., telescoping) over the liner (710), further configured to change the length of the needle obscuring assembly (700). As such, the needle obscuring assembly (700) includes an extended configuration and a retracted configuration, which will be described in further detail below. Moreover, the needle obscuring member (750) is configured to be disposed at least partially over the needle (620), as shown in FIG. 10F.

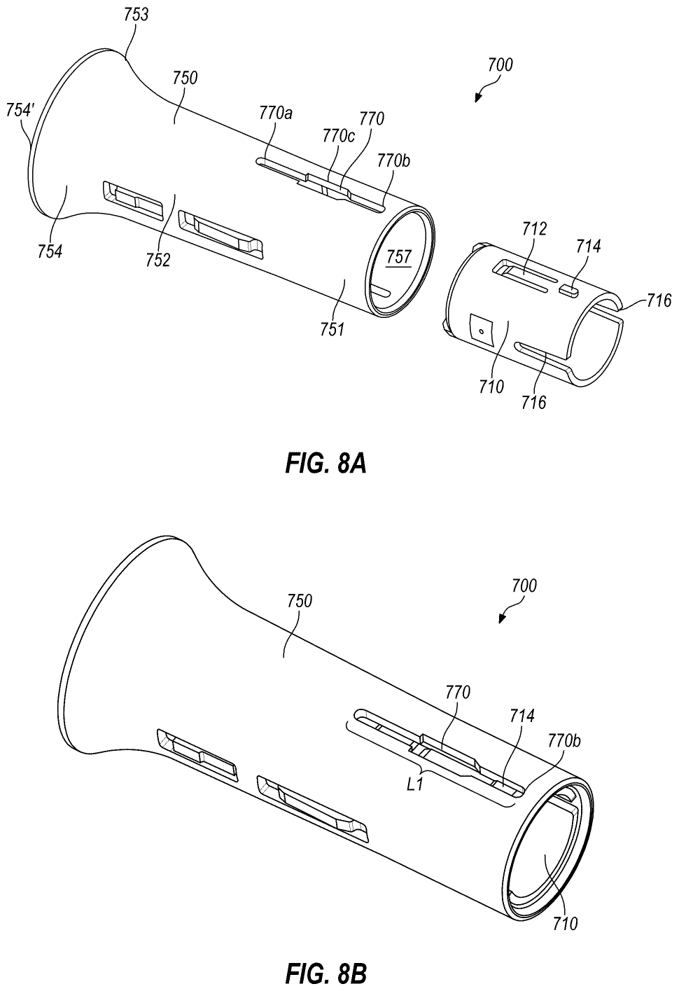

FIGS. 8A-8B illustrate the needle obscuring assembly (700), having the liner (710) and the needle obscuring member (750) pre-assembly (e.g., exploded view in FIG. 8A) and having the liner (710) and the needle obscuring member (750) assembled (e.g., coupled in FIG. 8B). The liner (710) includes a pair of protrusions (714), the pair of protrusions (714) extend outwardly from the liner (710). The liner (710) includes a substantially tubular configuration, where the pair of protrusions (714) extend radially outward and are disposed substantially opposite from each other (e.g., 180 degrees). In some embodiments, the pair of protrusions (714) may be disposed in a range between 160 to 200 degrees from each other. Each protrusion (714) is configured to be partially disposed within a respective slot (770) the needle obscuring member (750), as shown in FIG. 8B. The liner (710) further includes one or more slits (716), as shown in FIG. 8A. In some embodiments, the liner (710) includes a pair of slits (716) disposed substantially opposite from each other (e.g., 180 degrees). In some embodiments, the pair of slits (716) may be disposed in a range between 160 to 200 degrees from each other. Additionally, each of the slits (716) may be disposed substantially orthogonal (90 degrees) and/or in a range between 70 to 110 degrees from one of the protrusions (714). The slits (716) are configured to provide flexibility to the liner (710) for the needle obscuring assembly (700) to be formed or assembled. For example, when the needle obscuring member (750) is being disposed over the liner (710), at least a portion of the liner (710) is allowed to move radially inward until the pair of protrusions (714) are disposed within the respective slot (770) of the needle obscuring member (750).

As shown in FIG. 8A, the liner (710) further includes at least one flexible latch (712) configured to engage the annular surface (635) of the compression ring (630) and resist removal of the liner (710) from the needle hub (610). As shown in FIGS. 9B, 10A and 10B, a pair of flexible latches (712) of the liner (710) move radially outward (arrows “I” in FIGS. 9B and 10A) when the needle obscuring assembly (700) is translated proximately over the needle hub assembly (606), until the pair of flexible latches (712) aligned with the distal end (31) of the syringe body (34) and move radially inward (arrows “II” in FIGS. 9B and 10B) to engage the annular surface (635) of the compression ring (630) of the needle hub (610) securing the needle obscuring assembly (700) to the needle hub (610). Alternatively, the needle hub assembly (606) may be translated distally into the needle obscuring assembly (700) in FIGS. 9B, 10A and 10B; or simultaneous relative movement of the needle obscuring assembly (700) and the needle hub assembly (606) until the pair of flexible latches (712) snap in position therefore securing the needle obscuring assembly (700) to the needle hub (610). Further, the larger diameter of the compression ring (630) and/or the annular surface (635) with respect to the diameter of the syringe body (34) facilitates the engagement of the pair of flexible latches (712) and the securing of the needle obscuring assembly (700) to the needle hub (610).

As further shown in FIG. 8A, the needle obscuring member (750) includes a proximal end (751), a tubular body (752), a distal portion (754), and defines an annular space or lumen (757) extending between the proximal end (751) and the distal portion (754). The liner (710) of the needle obscuring assembly (700) is at least partially disposed in the annular space or lumen (757) of the needle obscuring member (750), as shown in FIGS. 8B-11.

Referring back to FIG. 8A, the distal portion (754) of the needle obscuring member (750) includes an outwardly sloping surface (753), such that the distal portion (754) has a larger diameter than the diameter of the tubular body (752) of the needle obscuring member (750). The outwardly sloping surface (753) of the distal portion (754) of the needle obscuring member (750) is configured to facilitate application of a distally directed force to the needle obscuring member (750) using the fingers and thumb of one hand of a user. These outwardly sloping surfaces (753) of the distal portion (754) of the needle obscuring member (750) is larger than the body portion (752), and therefore easier to handle than the smaller needle cover (760), thereby making the needle cover (760) removal easier. The distal portion (754) of the needle obscuring member (750) further includes a distally facing surface (754′). Additionally, the needle obscuring member (750) includes a proximally facing surface (770a) and a distally facing surface (770b) defining a respective slot (770). A pair of slots (770) extend longitudinally in the tubular body (752) of the needle obscuring member (750). In some embodiments, the pair of slots (770) are disposed adjacent to or in the proximity of the proximal end (751) of the needle obscuring member (750). In some embodiments, the pair of slots (770) include a wider portion or window (770c) configured to provide clearance for the latches (712) to move radially during assembly over the needle hub (610). The pair of slots (770) of the needle obscuring member (750) is configured to receive the respective protrusion (714) of the liner (710). As such, each protrusion (714) is partially disposed within the respective slot (770) and configured to interfere with the proximally facing surface (770a) of the needle obscuring member (750) to limit proximal movement of the needle obscuring member (750) relative to the liner (710) when the needle obscuring assembly (700) is moved into the retracted configuration (FIGS. 10G and 10H). Additionally, each protrusion (714) is further configured to interfere with the distally facing surface (770b) of the needle obscuring member (750) defining the respective slot (770) to limit distal movement of the needle obscuring member (750) relative to the liner (710) when the needle obscuring assembly (700) is moved into the extended configuration (FIGS. 8B, 9B). Therefore, when the needle obscuring member (750) is moved distally relative to the liner (710), substantially obscuring the needle (620), the needle obscuring assembly (700) is in the extended configuration (FIGS. 10E, 10F). Conversely, when the needle obscuring member (750) is moved proximally relative to the liner (710) at least partially exposing the needle (620), the needle obscuring assembly (700) is in the retracted configuration (FIGS. 10G and 10H). Further, each of the pair of slots (770) of the needle obscuring member (750) have a length “L1”, as shown in FIG. 8B. Length “L1 is configured to determine a corresponding length “L2” of the needle (620) when exposed (see FIG. 10G), which will be described in further detail below.

Alternatively, when the liner (710) and the needle obscuring member (750) substantially overlap, and the liner (710) is aligned with the proximal end (751) of the needle obscuring member (750), the needle obscuring assembly (700) is in the extended configuration (FIG. 10B). Additionally, when the needle obscuring member (750) is moved distally relative to the liner (710) and the liner (710) is not aligned with the proximal end (751) of the needle obscuring member (750), the needle obscuring assembly (700) is in the retracted configuration (FIGS. 10D and 10G). The needle obscuring assembly (700) is configured to move from a fully extended configuration (FIG. 10B) to a fully retracted configuration (FIG. 10G). Optionally, the needle obscuring assembly (700) is configured to move from a fully extended configuration (FIG. 10B) to a partially retracted configuration (FIG. 10D). Further, the needle obscuring assembly (700) is configured to move from a fully extended configuration to a fully (or partially) retracted configuration without locking in either configuration (i.e., the needle obscuring assembly (700) includes no locking system). Therefore, the needle obscuring assembly (700) is configured to freely move between the extended and retracted configurations, allowing the injection system (100) to be manipulated multiple times (e.g., needle obscuring assembly (700) may move back and forward between configurations). The needle obscuring assembly (700) is substantially the same length in the retracted and extended configurations (e.g., the length of the needle obscuring member (750)), since the needle obscuring member (750) translates relative to the liner (710) while the liner (710) is substantially covered by the needle obscuring member (750) in the retracted and extended configurations of the needle obscuring assembly (700).

As shown in FIGS. 9A and 10D-10H, the needle obscuring member (750) includes a plurality of inwardly extending movable members (755). The movable members (755) include latches, living hinges, flexible arms, fasteners, clasp, L-shape bar, hooks or any other movable, resistive and/or elastic material. The plurality of inwardly extending movable members (755) includes a first pair (755a) of inwardly extending movable members and a second pair (755b) of inwardly extending movable members. The first pair (755a) and second pair (755b) of inwardly extending movable members of the needle obscuring member (750) are each configured to movable radially inward and outward from the tubular body (752) of the needle obscuring member (750).

The injection system (100) further includes a needle cover (760), as shown in FIGS. 6-7, 9B and 10A-10E. The cover (760) is removably disposed within the lumen (757) of the needle obscuring member (750) and over the needle (620). As shown in FIGS. 9B, the needle cover (760) is disposed over a distal tip (621) of the needle (620) when the injection system (100) is in a transport/storage configuration. The distal tip (621) of the needle (620) includes sharp, beveled edge or others suitable shape configured to percutaneously penetrate the patient. As shown in FIGS. 9B, 10C-10D, the needle cover (760) includes a body surface (762), a proximally facing flange (764) and an interior region (766). The needle cover (760) may include an elastomeric material (768) in the interior region (766), the elastomeric sealing material (768) is configured to interface with the needle (620), the distal tip (621) and a distal housing portion of the needle (622), as shown in FIG. 9B. Further, the elastomeric material (768) of the needle cover (760) is configured to interfere with a circumferential groove (623) defined by the distal housing portion of the needle (622) or needle hub (610) to secure the needle cover (760) onto the needle hub (610). The elastomeric material (768) also protects the needle (620) from mechanical damage and seals the injection system (100) before use. In some embodiments, the needle cover (760) may optionally comprise a vent (761) for allowing pressure resulting from the transfer and/or mixing of the medicine components to escape from inside the syringe body (34) while preventing contamination from entering the syringe body (34). Moreover, the needle cover (760) minimizes accidental needle sticks during use.

FIG. 10C illustrate the needle obscuring member (750) moved proximally (750 arrow) relative to the liner (710) to at least partially expose the needle cover (760), so that the needle obscuring assembly (700) is moved to the retracted configuration. The plurality of inwardly extending movable members (755) of the needle obscuring member (750) are hidden from view behind the needle cover (760) in FIG. 10C, since the movable members (755) are in a different plane than the flexible latches (712) of the liner (710), as shown in FIG. 9A.

FIG. 10D illustrate the needle obscuring member (750) moved proximally relative to the liner (710), as shown in FIG. 10C, while depicting the plurality of inwardly extending movable members (755). As shown in FIG. 10D, the first pair (755a) of the inwardly extending movable members of the needle obscuring member (750) are configured to contact the body surface (762) of the needle cover (760). The first pair (755a) of the inwardly extending movable members of the needle obscuring member (750) is configured to exert a resistive force on the body surface (762) of the needle cover (760) to removably hold the position of the needle cover (760) relative to the needle obscuring assembly (700). The first pair (755a) of the inwardly extending movable members of the needle obscuring member (750) removably holds the position of the needle cover (760) when the needle obscuring assembly (700) is moved either in the retracted configuration (FIG. 10D) and/or in the extended configuration (FIG. 10E). However, the resistive force exerted on the needle cover (760) by the first pair (755a) of the inwardly extending movable members of the needle obscuring member (750) is configured to be overcome by a user's fingers to remove the needle cover (760) from the needle obscuring assembly (700), as shown in FIG. 10F, the needle cover (760) is removed.

As shown in FIGS. 10D and 10E, the second pair (755b) of the inwardly extending movable members of the needle obscuring member (750) is configured to contact the proximally facing flange (764) of the needle cover (760). The second pair (755b) of the inwardly extending movable members of the needle obscuring member (750) may include an arm-like configuration, having an arm (755b1), an elbow (755b2) and forearm (755b3) sections, as shown in FIG. 10D. The forearm (755b3) sections of the second pair (755b) of the inwardly extending movable members of the needle obscuring member (750) contacts the proximally facing flange (764) of the needle cover (760) and is disposed proximately to the needle cover (760) allowing the second pair (755b) of the inwardly extending movable members of the needle obscuring member (750) to push or translate the needle cover (760), as shown in FIG. 10E. Therefore, the needle obscuring member (750) is configured to move the needle cover (760) distally when the needle obscuring assembly (700) is moved distally into the extended configuration and exerts a distally directed force on the needle cover (760) through the second pair (755b) of the inwardly extending movable members of the needle obscuring member (750) to the proximally facing flange (764) of the needle cover (760), as shown in FIGS. 10D and 10E. As such, the second pair (755b) of the inwardly extending movable members of the needle obscuring member (750) are configured to exert a distally directed force from the needle obscuring member (750) to proximally facing flange (764) of the needle cover (760), thereby removing the needle cover (760) from the needle hub (610). The distally directed force transferred from the needle obscuring member (750) through second pair (755b) of the inwardly extending movable members is sufficient to overcome the resistive force between the elastomeric material (768) of the needle cover (760) and the outside surface of the needle hub (610) including the circumferential groove (623) form therein. The second pair (755b) of the inwardly extending movable members are “self-energizing” in that as the second pair (755b) of the inwardly extending movable members are pushed distally and contact the needle cover (760), increasing the distally directed force applied to the second pair (755b) of the inwardly extending movable members increases a radially inward force moving the second pair (755b) of the inwardly extending movable members into engagement with the needle cover (760).

When the needle obscuring assembly (700) is moved into the extended configuration and the needle cover (760) is moved distally, as shown in FIG. 10E, the force required to remove the needle cover (760) from the needle hub (610) is reduced or minimized. When the needle cover (760) is moved distally by the needle obscuring assembly (700), the elastomeric material (768) of the needle cover (760) (FIG. 9B) no longer interferes with the circumferential groove (623) defined by the needle hub (610) (FIG. 10E), reducing the force required to remove the needle cover (760) from the needle hub (610). At the same time, the elastomeric material (768) of the needle cover (760) is moved away from the distal tip (621) of the needle (620) such that the distal tip (621) is no longer closed by the elastic material (632). As such, any excess pressure built up in the syringe body (34) can be vented through the needle (620). Holding the injection system (100) in a vertical position during venting avoids loss of injectable fluid while allowing gas and pressure to vent. After optional venting, the user can gently pull the needle cover (760) from the injection system (100) to prepare for an injection.

While the needle obscuring assembly (700) is described as facilitating removal of the needle cover (760) from the needle hub (610) utilizing distally directed force applied to needle cover (760), a user may remove the needle cover (760) from the needle hub (610) by pulling the needle cover (760), which is accessible to the user even with the needle obscuring assembly (700) in extended configuration (FIGS. 10A-10B). However, most of the access to the needle cover (760) is blocked by needle obscuring assembly (700) in the extended configuration (FIGS. 10A-10B), thereby discouraging users from pulling off the needle cover (760) and encouraging users to use the needle obscuring assembly (700) to loosen the needle cover (760). Although users may remove the needle cover (760) manually using their hands, when the needle obscuring assembly (700) is in the retracted configuration, the needle obscuring assembly (700) still covers the distal tip (621) of the needle (620). This makes the needle cover (760) removal safer and easier because the user's hands are protected against contact with the needle (620) during removal and protects the needle (620) from being bent or damaged.

As shown in FIG. 10F, the needle cover (760) is removed from the needle hub (610) while the needle obscuring assembly (700) is in the extended configuration substantially obscuring, concealing, and/or hiding the needle (620) and the distal tip (621) of the needle (620). As shown in FIGS. 10F-10H, the second pair (755b) of inwardly extending movable members of the needle obscuring member (750) is configured to move radially outward from the tubular body (752) of the needle obscuring member (750) when the needle obscuring assembly (700) is in the retracted configuration partially exposing the needle (620). Further, the first pair (755a) of inwardly extending movable members of the needle obscuring member (750) is configured to stay in the radially inward position from the tubular body (752) of the needle obscuring member (750) when the needle obscuring assembly (700) is in the retracted configuration, partially exposing the needle (620).

Additionally, the distal portion (754) of the needle obscuring member (750) prevents the distal tip (621) of the needle from being seen from proximal of the needle obscuring member (750) when the needle obscuring assembly (700) is in the extended configuration, as shown in FIG. 10F. The distal portion (754) of the needle obscuring member (750) (e.g., distally facing surface 754′) is configured to contact a patient's skin (50). In some embodiments, the distal portion (754) of the needle obscuring member (750) is configured to contact a patient's skin (50) before a distal tip (621) of the needle (620) contact the patient's skin (50) during an injection using the system (100) when needle obscuring assembly (700) is in the extended configuration. The distal tip (621) of the needle (620) is obscured, concealed, or hidden by the needle obscuring member (750) in the extended configuration, such that when the distal portion (754) of the needle obscuring member (750) contacts and/or moves across the patient's skin (50), the patient will not be scratched by the distal tip (621) of the needle (620).

The needle obscuring assembly (700) is pushed back or translated in a proximal direction when the distal portion (754) of the needle obscuring member (750) is pushed or pressed against the patient's skin (50) and the needle obscuring assembly (700) is in the retracted configuration, while still obscuring viewing of the needle (620). As such, the distal tip (621) of the needle (620) is hidden (i.e., not visible from proximal of the needle obscuring member (750)). This reduces anxiety in a patient receiving an injection by obscuring the needle (620) and the distal tip (621) thereof from the patient's vision. Further, the distally facing surface (754′) of the needle obscuring member (750) makes contact completely around the injection site before the distal tip (621) of the needle (620). In some embodiments, the distally facing surface (754′) of the needle obscuring member (750) contacts the patient's skin shortly before (e.g., about 1 second) the distal tip (621) of the needle (620). As such, the contact around the injection site hides/reduces/obscures the sense of pain from the injection with the distal tip (621) of the needle (620) by providing different tactile sensations to the nerves around the injection site. The injection angle of the injection system (100) with the needle obscuring assembly (700) installed may be optimally restricted to substantially orthogonal. Substantially orthogonal includes a maximum of 15° to 30° from orthogonal.

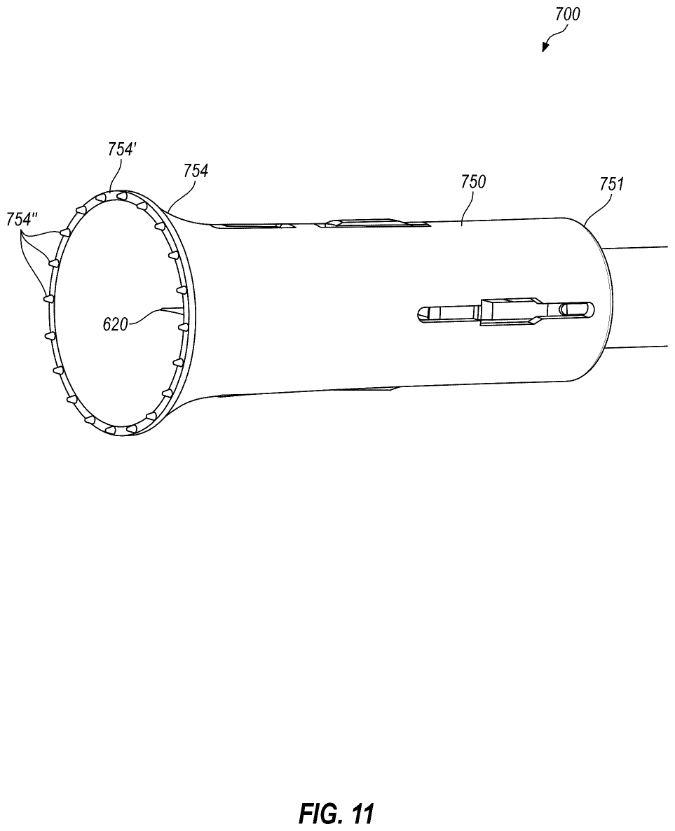

Additionally, the outwardly sloping surface (753) of the distal portion (754) of the needle obscuring member (750) is configured to facilitate application of a distally directed force to the needle obscuring member (750), as shown by arrows in FIG. 10G. The injection system (100) is ready for injection after removal of the needle cover (760) with the needle obscuring assembly (700) in the retracted configuration (FIG. 10G). The distally facing surface (754′) of the distal portion (754) of the needle obscuring member (750) includes a substantially flat surface (FIG. 10F). In some embodiments, the distal portion (754) of the needle obscuring member (750) comprises a plurality of protrusions or cones (754″) on a distally facing surface (754′) (FIG. 11). The plurality of protrusions or cones (754″) are configured to provide sensory masking effect that minimizes or reduce injection pain in patients (e.g., sending confusing mild pain signals, masking the pain from the needle).

Additionally, the length “L1” (FIG. 8B) of each of the pair of slots (770) of the needle obscuring member (750) determine the corresponding predetermined length “L2” of the needle (620) when exposed (see FIG. 10G). Since each protrusion (714) of the liner (710) disposed within the respective slot (770) of the needle obscuring member (750) interferes with the proximally facing surface (770a) of the slot (770) to limit proximal movement of the needle obscuring member (750) relative to the liner (710) when the needle obscuring assembly (700) is moved into the retracted configuration (FIGS. 10D, 10G, 10H). Thus, the length “L1” of each of the slots (770) limits the travel distance of the protrusion (714) of the liner (710) and therefore limits the amount of exposed length “L2” of the needle (620) when the needle obscuring assembly (700) is in the retracted configuration, as shown in FIG. 10G. As such, the length “L1” of the slot (770) of the needle obscuring member (750), as shown in FIG. 8B, determines how much of the needle (620) is exposed (i.e., predetermined length “L2” shown in FIG. 10G). The predetermined length “L2” of the needle (620) is in a range between 25 to 6 millimeters. In some embodiments, the predetermined length “L” of the needle (620) is about 8 millimeters. In some embodiments, the length “L1” of the slot (770) may be directly proportional to the predetermined length “L2” of the needle (620). A desirable predetermined length “L2” may be easily achieved by adjusting the length “L1” of the slot (770) during the manufacturing process of the needle obscuring member (750).

The injection system (100) further includes a safe retraction system configured to retract the needle (620) at least partially within the syringe body (34) after an injection with the system is completed such that the distal tip (621) of the needle is disposed within the needle hub (610) and/or the syringe body (34), such as those described in U.S. patent application Ser. No. 18/300,125, which was previously incorporated by reference herein. Therefore, there is no need for the needle obscuring assembly (700) to be disposed in the extended configuration (e.g., distal position obscuring the needle), after the injection of a patient is performed. The needle obscuring assembly (700) is free to move from the retracted configuration (e.g., proximal position) into the extended configuration (e.g., distal position) and vice versa, before, during and after the injection with the system (100) is performed. The needle obscuring assembly (700) does not include any locking mechanism and does not lock in either the retracted or extended configuration. The needle obscuring assembly (700) does not function as a needle cover or shield, instead the needle obscuring assembly (700) is configured to obscure, hid, block or minimize the view of the needle (620) by the patient. Thus, the needle obscuring assembly (700) obscure a patient's view of the needle (620) piercing the skin (50) reducing or obscuring the sense of pain from the injection with the distal tip (621) of the needle (620) by providing different tactile sensations. As such, the needle obscuring assembly (700) both obscures a patient's view of the needle (620) piercing the skin (50) and reduces/obscures the sense of pain from the injection with the distal tip (621) of the needle (620) by providing different tactile sensations to the nerves around the injection site (e.g., plurality of protrusions or cones (754″).

The embodiments described herein include unvented single chamber, vented dual chamber, and unvented dual chamber injection systems.

While the embodiments described above include single and dual chamber (safety) injection systems, the scope of the claims also include other multiple chamber injection systems, with or without safe injection systems. For multiple chamber safety injection systems with more than two chambers, more than two stopper members are inserted into an injection system body (e.g., syringe body, cartridge body, etc.) to define a corresponding number of chambers.

While the injection systems depicted and described herein include syringes with staked needles, the needle cover handling devices/telescoping members described herein can be used with cartridges, an auto injector, and injection systems with Luer connectors, etc. Any suitable manufacturing technique (e.g., molded unitary elements, molded pieces coupled together, laser cut, 3D laser printing, etching, etc.) may be used for the system and components described herein.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, PTFE, ETFE, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

1. A system for injection, comprising:
a syringe body having proximal and distal ends and defining a syringe interior;
a stopper member disposed in the syringe interior;
a plunger member coupled to the stopper member and configured to be manipulated to insert the stopper member distally in the syringe interior relative to the syringe body;
a needle hub assembly coupled to the syringe body at the distal end thereof and comprising
a needle hub, and
a needle coupled to the needle hub;
a needle obscuring assembly removably coupled to the needle hub over the needle and comprising;
a liner coupled to the needle hub, and
a needle obscuring member slidably disposed on and movable relative to the liner, the needle obscuring member configured to be disposed at least partially over the needle; and
a safe retraction system configured to retract the needle at least partially within the syringe body after an injection with the system is completed such that a distal tip of the needle is disposed within the needle hub or the syringe body.

2. The system of claim 1, wherein the needle hub comprises a compression ring, the compression ring defines an annular surface.

3. The system of claim 2, wherein the liner comprises a flexible latch configured to engage the annular surface of the compression ring and resist removal of the liner from the needle hub.

4. The system of claim 3, wherein the latch of the liner is further configured to secure the needle obscuring assembly to the needle hub.

5. The system of claim 1, wherein the needle obscuring assembly comprises an extended configuration and a retracted configuration,
wherein the needle obscuring member is moved distally relative to the liner, substantially obscuring the needle, when the needle obscuring assembly is in the extended configuration, and
wherein the needle obscuring member is moved proximally relative to the liner at least partially exposing the needle, when the needle obscuring assembly is in the retracted configuration.

6. The system of claim 5, wherein the liner comprises a pair of protrusions,
wherein the needle obscuring member defines a pair of slots, and
wherein each protrusion is partially disposed within a respective slot and configured to interfere with a proximally facing surface of the needle obscuring member defining the respective slot to limit proximal movement of the needle obscuring member relative to the liner when the needle obscuring assembly is moved into the retracted configuration.

7. The system of claim 6, wherein each protrusion is further configured to interfere with a distally facing surface of the needle obscuring member defining the respective slot to limit distal movement of the needle obscuring member relative to the liner when the needle obscuring assembly is moved into the extended configuration.

8. The system of claim 5, wherein the needle obscuring member comprises a proximal end, a tubular body, a distal portion,
wherein the needle obscuring member defines a lumen extending between the proximal end and the distal portion, and
wherein the tubular body comprises a plurality of inwardly extending movable members.

9. The system of claim 8, wherein the plurality of inwardly extending movable members comprises a first pair of inwardly extending movable members and a second pair of inwardly extending movable members.

10. The system of claim 9, wherein the first pair and second pair of inwardly extending movable members of the needle obscuring member are each movable radially inward and outward from the tubular body of the needle obscuring member.

11. The system of claim 10, wherein the system further comprises a needle cover removably disposed within the lumen of the needle obscuring member and over the needle, wherein the needle cover comprises a body surface and a proximally facing flange, and wherein the first pair of the inwardly extending movable members of the needle obscuring member is configured to contact the body surface of the needle cover and the second pair of the inwardly extending movable members of the needle obscuring member is configured to contact the proximally facing flange of the needle cover.

12. The system of claim 11, wherein the needle obscuring member is configured to move the needle cover distally when the needle obscuring assembly is moved distally into the extended configuration and exerts a distally directed force on the needle cover through the second pair of the inwardly extending movable members of the needle obscuring member to the proximally facing flange of the needle cover.

13. The system of claim 12, wherein the first pair of the inwardly extending movable members of the needle obscuring member is configured to exert a resistive force on the body surface of the needle cover to removably hold the position of the needle cover relative to the needle obscuring assembly, when the needle obscuring assembly is moved into the extended configuration.

14. The system of claim 13, wherein resistive force exerted on the needle cover by the first pair of the inwardly extending movable members of the needle obscuring member is configured to be overcome by a user's fingers to remove the needle cover from the needle obscuring assembly.

15. The system of claim 12, wherein moving the needle cover distally as the needle obscuring assembly is moved into the extended configuration reduces a force required to remove the needle cover from the needle hub.

16. The system of claim 10, wherein the first pair of inwardly extending movable members of the needle obscuring member is configured to move radially outward from the tubular body of the needle obscuring member when the needle obscuring assembly is in the retracted configuration to partially expose the needle.

17. The system of claim 10, wherein the second pair of inwardly extending movable members of the needle obscuring member is configured to move radially inward from the tubular body of the needle obscuring member when the needle obscuring assembly is in the retracted configuration to partially expose the needle.

18.-22. (canceled)

23. The system of claim 8, wherein the distal portion of the needle obscuring member prevents a distal tip of the needle from being seen from proximal of the needle obscuring member when the needle obscuring assembly is in the extended configuration.

24. The system of claim 8, wherein the distal portion of the needle obscuring member is configured to contact a patient's skin before a distal tip of the needle contacts the patient's skin during an injection using the system when needle obscuring assembly is in the extended configuration.

25. (canceled)

26. The system of claim 8, wherein the distal portion of the needle obscuring member comprises a plurality of protrusions or cones on a distally facing surface thereof.

27.-34. (canceled)
